(19) United States
(12) Patent Application Publication
(10) Pub. No.: US 2022/0184343 A1
(43) Pub. Date: Jun. 16, 2022

(54) CATHETER SYSTEM, CATHETER ASSEMBLY, AND CATHETER GRIPPER

(71) Applicant: TERUMO KABUSHIKI KAISHA, Tokyo (JP)

(72) Inventors: Shinichi MIZUNO, Nakakoma-gun (JP); Shinnosuke YAMASHITA, Ljubljana (SI)

(73) Assignee: TERUMO KABUSHIKI KAISHA, Tokyo (JP)

(21) Appl. No.: 17/683,991

(22) Filed: Mar. 1, 2022

Related U.S. Application Data
(63) Continuation of application No. PCT/JP2020/033401, filed on Sep. 3, 2020.

(30) Foreign Application Priority Data
Sep. 9, 2019 (JP) 2019-163524

(51) Int. Cl. A61M 25/00; A61M 25/06

(52) U.S. Cl. CPC A61M 25/0084; A61M 25/0097; A61M 2205/7536; A61M 2025/0089; A61M 25/0625

(57) ABSTRACT

A catheter system includes: a catheter assembly that includes: an inner needle having a needle tip at a distal end thereof, a hollow needle hub fixed to a proximal end section of the inner needle, a catheter shaft into which the inner needle is inserted, and a catheter hub having the inner needle inserted thereinto, wherein the catheter hub is fixed to a proximal end section of the catheter shaft; and a catheter gripper that is attachable to and detachable from the catheter assembly, wherein the catheter gripper includes: a gripping member, and an attaching/detaching mechanism configured to attach and detach a proximal end section of the needle hub to and from a first end section of the gripping member.

### CROSS-REFERENCE TO RELATED APPLICATIONS

This application is a bypass continuation of PCT Application No. PCT/JP2020/033401, filed Sep. 3, 2020, which claims priority to Japanese Application No. 2019-163524, filed on Sep. 9, 2019. The contents of these applications are hereby incorporated by reference in their entirety.

### BACKGROUND

The present disclosure relates to a catheter system, a catheter assembly, and a catheter gripper.

JP 2012-71052 A discloses a catheter assembly for administrating an infusion solution into a patient. The catheter assembly includes an inner needle having a needle tip at a distal end thereof, a needle hub fixed to a proximal end section of the inner needle, a catheter shaft into which the inner needle is inserted, and a catheter hub having the inner needle thereinto and fixed to a proximal end section of the catheter shaft.

### SUMMARY

In the above-described catheter assembly, the needle hub is formed long enough to be gripped by an operator. Therefore, there is a problem that an overall length of the catheter assembly becomes long, a storage space increases, and the used catheter assembly (waste) also increases in size.

Embodiments of the present invention have been developed in view of such problems, and an object of certain embodiments of the present invention is to provide a catheter system, a catheter assembly, and a catheter gripper capable of reducing a storage space and reducing a size of waste.

According to a first aspect of the present invention, a catheter system includes a catheter assembly that includes an inner needle having a needle tip at a distal end thereof, a hollow needle hub fixed to a proximal end section of the inner needle, a catheter shaft into which the inner needle is inserted, and a catheter hub having the inner needle inserted thereinto and fixed to a proximal end section of the catheter shaft, and a catheter gripper that is attachable to and detachable from the catheter assembly, in which the catheter gripper includes a gripping member that extends in one direction, and an attaching/detaching mechanism that attaches/detaches a proximal end section of the needle hub to/from a first end section of the gripping member.

According to a second aspect of the present invention, a catheter assembly includes an inner needle that has a needle tip at a distal end thereof, a hollow needle hub that is fixed to a proximal end section of the inner needle, a catheter shaft into which the inner needle is inserted, and a catheter hub that has the inner needle inserted thereinto and is fixed to a proximal end section of the catheter shaft, in which a proximal end section of the needle hub is provided with a mounting section for mounting on a catheter gripper.

According to a third aspect of the present invention, a catheter gripper attachable to and detachable from a catheter assembly that includes an inner needle that has a needle tip at a distal end thereof, a hollow needle hub that is fixed to a proximal end section of the inner needle, a catheter shaft into which the inner needle is inserted, and a catheter hub that has the inner needle inserted thereinto and is fixed to a proximal end section of the catheter shaft, the catheter gripper includes a gripping member that extends in one direction, and an attaching/detaching mechanism that attaches/detaches a proximal end section of the needle hub to/from a first end section of the gripping member.

According to certain embodiments of the present invention, because a catheter assembly can be attached to and detached from a catheter gripper, the catheter assembly can be configured as a disposable product and the catheter gripper can be configured as a reusable product. As a result, it is not necessary to provide a gripping portion in a catheter assembly to be discarded after one use and, thus, an entire length of the catheter assembly can be made relatively short. Therefore, the waste (catheter assembly) can be reduced in size. In addition, because the number of catheter grippers, which are reusable products, is usually less than the number of catheter assemblies, which are disposable products, storage space can be reduced.

### BRIEF DESCRIPTION OF THE DRAWINGS

FIG. 1 is a perspective view of a catheter system according to an embodiment of the present invention;

FIG. 2 is an exploded perspective view of a catheter system of FIG. 1;

FIG. 3 is a longitudinal sectional view of a catheter assembly of FIG. 2;

FIG. 4 is a longitudinal sectional view of a catheter gripper of FIG. 2;

FIG. 5A is a first cross-sectional explanatory view for explaining an operation of mounting the catheter assembly on the catheter gripper, and FIG. 5B is a second cross-sectional explanatory view for explaining the operation of mounting the catheter assembly on the catheter gripper;

FIG. 6 is a third cross-sectional explanatory view for explaining the operation of mounting the catheter assembly on the catheter gripper;

FIG. 7 is a first cross-sectional explanatory view for explaining an operation of removing the catheter assembly from the catheter gripper; and

FIG. 8 is a second cross-sectional explanatory view for explaining the operation of removing the catheter assembly from the catheter gripper.

### DETAILED DESCRIPTION

Hereinafter, embodiments of a catheter system, a catheter assembly, and a catheter gripper according to the present invention will be described with reference to the accompanying drawings.

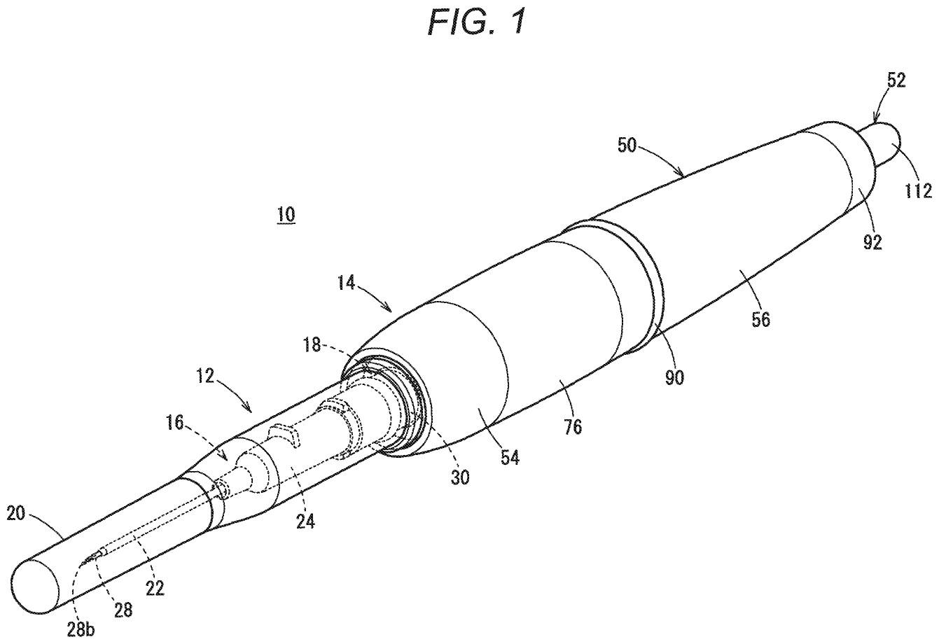

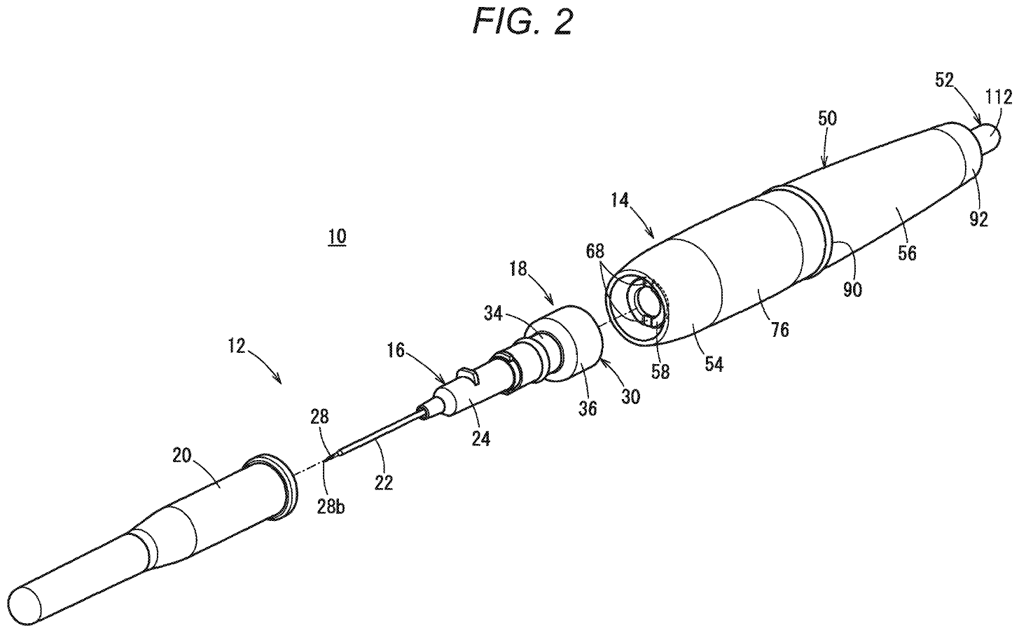

A catheter system 10 according to an embodiment of the present invention is configured as an indwelling needle for administering an infusion solution (medicinal solution) into a blood vessel of a patient (living body). As illustrated in FIGS. 1 and 2, the catheter system 10 includes a catheter assembly 12 that is a disposable product, and a catheter gripper 14 that is a reusable product that can be repeatedly used.

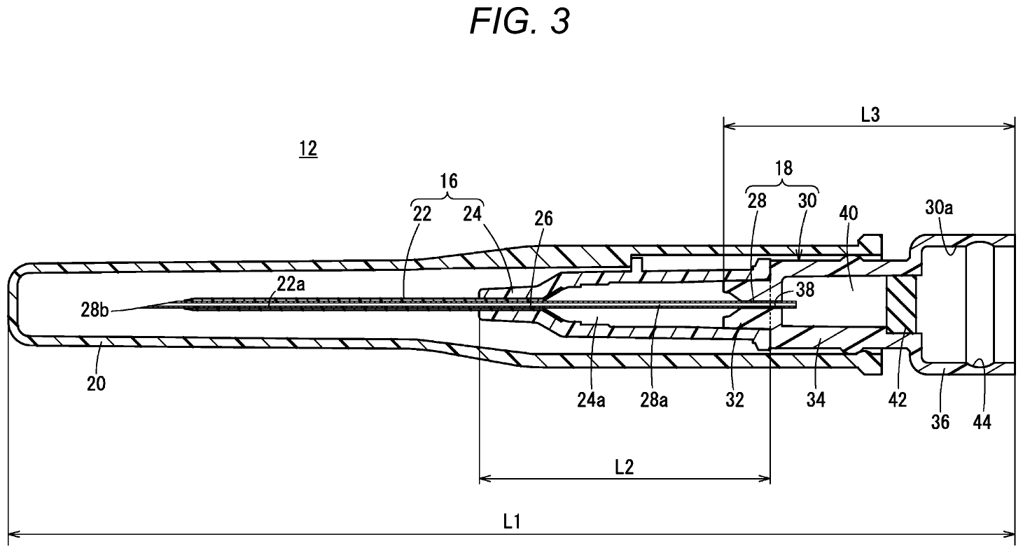

In FIG. 2, the catheter assembly 12 is detachable from the catheter gripper 14. The catheter assembly 12 includes a catheter body 16, a needle member 18, and a protector 20. As illustrated in FIG. 3, the catheter body 16 includes a catheter shaft 22 and a catheter hub 24 fixed to a proximal end section of the catheter shaft 22.

The catheter shaft 22 is a tubular member having flexibility and capable of being continuously inserted into a blood vessel of a patient. The catheter shaft 22 has an inner cavity 22a extending along an axial direction over the entire length thereof.

Although a constituent material of the catheter shaft 22 is not particularly limited, a resin material having transparency, particularly, a soft resin material is preferable. Examples thereof include a fluorine-based resin such as polytetrafluoroethylene (PTFE), an ethylene-tetrafluoroethylene copolymer (ETFE), and perfluoroalkoxy fluorine resin (PFA), an olefin-based resin such as polyethylene and polypropylene, or a mixture thereof, polyurethane, polyester, polyamide, polyether nylon resin, a mixture of an olefin-based resin and an ethylene-vinyl acetate copolymer, and the like.

The catheter hub 24 is formed in a hollow shape (cylindrical shape). A distal end section of the catheter hub 24 is provided with a first attachment hole 26 to which a proximal end section of the catheter shaft 22 is attached. An outer peripheral surface of the proximal end section of the catheter shaft 22 is fixed to a wall surface forming the first attachment hole 26 by an appropriate fixing means such as caulking, fusion, or adhesion.

An inner cavity 24a communicating with the inner cavity 22a of the catheter shaft 22 is formed on a proximal end side of the catheter hub 24 with respect to the first attachment hole 26. The inner cavity 24a of the catheter hub 24 is open to the base end of the catheter hub 24. Although not illustrated, a hemostasis valve, a seal member, and a plug may be disposed in the inner cavity 24a of the catheter hub 24.

The catheter hub 24 is preferably made of a material harder than the catheter shaft 22. Although a constituent material of the catheter hub 24 is not particularly limited, for example, thermoplastic resins such as polypropylene, polycarbonate, polyamide, polysulfone, polyarylate, a methacrylate-butylene-styrene copolymer, polyurethane, an acrylic resin, and an ABS resin can be preferably used.

The needle member 18 includes an inner needle 28 and a needle hub 30 fixed to a proximal end section of the inner needle 28. The inner needle 28 is a tubular member having rigidity capable of puncturing a skin of a patient. The inner needle 28 has an inner cavity 28a extending along an axial direction over the entire length thereof. The inner needle 28 is inserted into the inner cavity 22a of the catheter shaft 22 and the inner cavity 24a of the catheter hub 24 in an assembled state of the catheter assembly 12.

Examples of the constituent material of the inner needle 28 include metal materials such as stainless steel, aluminum, an aluminum alloy, titanium, and a titanium alloy. The inner needle 28 is formed sufficiently longer than the catheter shaft 22, and a sharp needle tip 28b (tip of the inner needle 28) protrudes from a distal end opening of the catheter shaft 22 in the assembled state of the catheter assembly 12.

The needle hub 30 is formed in a hollow shape (cylindrical shape). The constituent material of the needle hub 30 may be the same as that of the catheter hub 24 described above. The needle hub 30 includes an inner needle support section 32 constituting a distal end section thereof, a needle hub body 34 extending from the inner needle support section 32 to the proximal end side, and a mounting section 36 provided at a proximal end section of the needle hub body 34.

A second attachment hole 38 to which the proximal end section of the inner needle 28 is attached is formed in the inner needle support section 32. An outer peripheral surface of the proximal end section of the inner needle 28 is fixed to a wall surface forming the second attachment hole 38 by an appropriate fixing means such as fusion, adhesion, and fitting. The inner needle support section 32 is inserted inside the proximal end section of the catheter hub 24 in the assembled state of the catheter assembly 12.

The needle hub body 34 is provided with a chamber 40 that communicates with the inner cavity 28a of the inner needle 28 and into which blood guided from the inner needle 28 flows. The chamber 40 forms a part of the inner cavity 30a of the needle hub 30. At least a part (needle hub body 34) of the needle hub 30 has transparency so that the blood flowing into the chamber 40 can be visually recognized from the outside of the needle hub 30. That is, the needle hub 30 is made of a transparent or translucent material.

The chamber 40 is provided with a filter member 42 that allows air to flow but blocks blood from flowing. The filter member 42 is fixed to an inner surface of the proximal end section of the needle hub body 34.

The mounting section 36 is an annular portion mounted on the catheter gripper 14 (see FIG. 2) and extends in a proximal end direction from the proximal end of the needle hub body 34. The entire length of the mounting section 36 along the axial direction of the needle member 18 is shorter than the entire length of the needle hub body 34. An inner diameter of the mounting section 36 is greater than that of the needle hub body 34 adjacent to a distal end side of the mounting section 36. That is, a step is formed between the inner surface of the needle hub body 34 and the inner surface of the mounting section 36. An annular locking groove 44 extending along a circumferential direction of the mounting section 36 is formed on an inner peripheral surface of the mounting section 36. The locking groove 44 is formed by a groove wall surface having an arcuate cross section. The locking groove 44 is positioned on a distal end side with respect to a proximal end of the mounting section 36.

The protector 20 is a cylindrical protective member that covers the needle tip 28b of the inner needle 28 in a state (initial state) before use of the catheter assembly 12. In the initial state of the catheter assembly 12, the inner needle 28 and the catheter shaft 22 are housed in the distal end side of the inner cavity of the protector 20. In the initial state of the catheter assembly 12, a proximal end side of the protector 20 is fitted to the catheter hub 24 and the needle hub 30. Note that, in the initial state of the catheter assembly 12, a proximal end of the protector 20 is positioned on the distal end side (an outer peripheral side of the needle hub body 34) of the mounting section 36.

The entire length L1 of the catheter assembly 12 is preferably set to 6 cm or more and 10 cm or less, more preferably set to 7 cm or more and 9 cm or less, and still more preferably set to about 8 cm. A ratio of an entire length L3 of the needle hub 30 to an entire length L2 of the catheter hub 24 is preferably 150% or less, more preferably 120% or less, and still more preferably about 100%. In the present embodiment, the entire length L3 of the needle hub 30 is substantially the same as the entire length L2 of the catheter hub 24. Specifically, each of the entire length L3 of the needle hub 30 and the catheter hub 24 is preferably set to 2 cm or more and 4 cm or less, and more preferably set to about 2.5 cm.

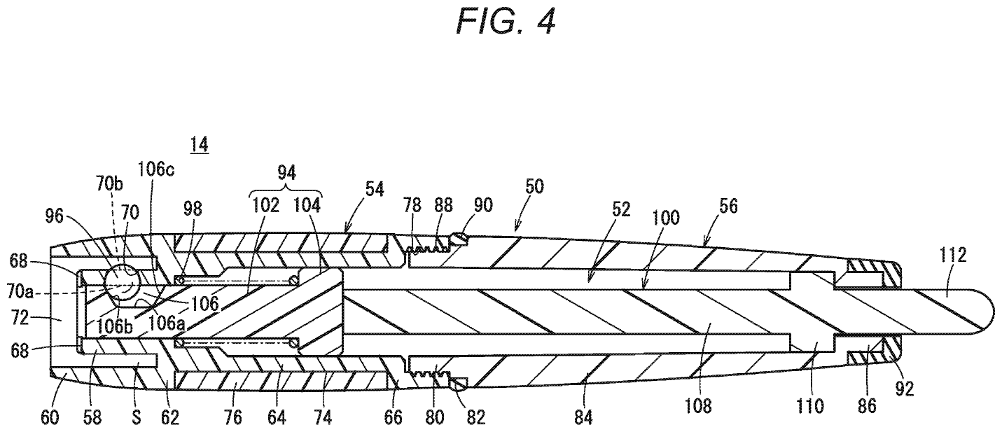

As illustrated in FIG. 2, the catheter gripper 14 is used together with the catheter assembly 12. The catheter gripper 14 is formed so that the catheter assembly 12 is attachable/detachable. In FIG. 4, the catheter gripper 14 includes a gripping member 50 extending in one direction and an attaching/detaching mechanism 52 provided on the gripping member 50. The gripping member 50 is formed to have an appropriate size (thickness, length) so as to be easily gripped and operated by the user.

The gripping member 50 includes a first cylinder member 54 positioned on the distal end side and a second cylinder member 56 connected to the proximal end side of the first cylinder member 54. Each of the first cylinder member 54 and the second cylinder member 56 is made of a metal material, a hard resin material, or the like. Examples of the hard resin material include the same materials as those of the catheter hub 24 described above.

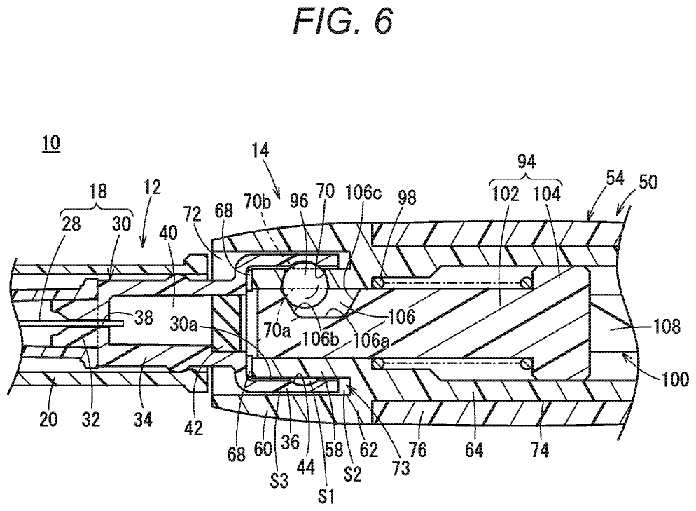

The first cylinder member 54 includes an inner cylinder section 58, an outer cylinder section 60, a connection section 62, an intermediate cylinder section 64, and a proximal end cylinder section 66. The inner cylinder section 58 is inserted into the inside of the mounting section 36 of the catheter assembly 12 (see FIG. 6). In FIGS. 2 and 4, two air discharge grooves 68 are formed on a distal end surface of the inner cylinder section 58. Each air discharge groove 68 extends along a radial direction from an inner peripheral surface to an outer peripheral surface of the inner cylinder section 58. The two air discharge grooves 68 are provided at positions shifted by 180° in the circumferential direction of the inner cylinder section 58. However, a size, a position, a quantity, and a shape of the air discharge groove 68 can be appropriately changed.

In FIG. 4, a hole 70 penetrating from the inner peripheral surface to the outer peripheral surface of the inner cylinder section 58 is formed at an intermediate section in the axial direction of the inner cylinder section 58. An inner opening 70a of the hole 70 that is opened to the inner peripheral surface of the inner cylinder section 58 is formed in a circular shape. An outer opening 70b of the hole 70 that is opened to the outer peripheral surface of the inner cylinder section 58 is formed in a circular shape. The diameter of the outer opening 70b is less than that of the inner opening 70a. That is, the diameter of the hole 70 gradually decreases from the inner peripheral surface toward the outer peripheral surface of the inner cylinder section 58.

The outer cylinder section 60 is provided on the outer peripheral side of the inner cylinder section 58. A gap S into which the mounting section 36 (see FIG. 6) is inserted is formed between the inner cylinder section 58 and the outer cylinder section 60. The distal end of the outer cylinder section 60 is positioned in the distal end direction with respect to the distal end of the inner cylinder section 58. The mounting hole 72, opened at the distal end and into which the mounting section 36 (see FIG. 6) of the catheter assembly 12 is inserted, is formed inside the outer cylinder section 60. That is, the mounting hole 72 is formed at the distal end section (first end section) of the gripping member 50. The outer surface of the outer cylinder section 60 is reduced in diameter in the distal end direction.

The connection section 62 connects a proximal end section of the inner cylinder section 58 and a proximal end section of the outer cylinder section 60 to each other. The intermediate cylinder section 64 extends in the proximal end direction from the connection section 62. An outer diameter of the intermediate cylinder section 64 is less than that of the connection section 62. An inner diameter of the intermediate cylinder section 64 is greater than that of the inner cylinder section 58.

The proximal end cylinder section 66 extends in a proximal end direction from a proximal end section of the intermediate cylinder section 64. The outer diameter of the proximal end cylinder section 66 is greater than that of the intermediate cylinder section 64. In other words, the outer diameter of the proximal end cylinder section 66 is substantially the same as that of the connection section 62. That is, an annular groove 74 having an outer peripheral surface of the intermediate cylinder section 64 as a bottom surface is formed between the connection section 62 and the base end cylinder section 66.

An annular non-slip member 76 is disposed in the annular groove 74. The non-slip member 76 is made of, for example, a resin material such as rubber. An outer peripheral surface of the non-slip member 76 is smoothly connected to an outer peripheral surface of the connection section 62 and an outer peripheral surface of the proximal end cylinder section 66 without a step. An inner peripheral surface of the proximal end cylinder section 66 is provided with a female screw section 78.

As illustrated in FIG. 6, the first cylinder member 54 (gripping member 50) is provided with an air vent flow path 73 for discharging air, which is guided from the inner cavity 30a (chamber 40) of the needle hub 30 to the gripping member 50 side, to the outside in a state in which the mounting section 36 of the catheter assembly 12 is mounted on the mounting hole 72. The air vent flow path 73 includes an air discharge groove 68, a first gap S1 between the inner cylinder section 58 and the mounting section 36, a second gap S2 between the mounting section 36 and the connection section 62, and a third gap S3 between the mounting section 36 and the outer cylinder section 60.

In FIG. 4, the second cylinder member 56 includes an insertion cylinder section 80, a stepped section 82, a gripping section main body 84, and a proximal end reduced diameter section 86. The insertion cylinder section 80 is inserted into the proximal end cylinder section 66. An outer peripheral surface of the insertion cylinder section 80 is provided with a male screw section 88 screwed into the female screw section 78.

The stepped section 82 protrudes radially outward from the proximal end section of the insertion cylinder section 80 and extends annularly in the circumferential direction. The stepped section 82 abuts on the proximal end surface of the proximal end cylinder section 66 in a state in which the female screw section 78 and the male screw section 88 are completely screwed. A protruding length of the stepped section 82 radially outward from the insertion cylinder section 80 is less than a thickness of the insertion cylinder section 80.

The gripping section main body 84 is formed in a cylinder shape and extends in the proximal end direction from the stepped section 82. The stepped section 82 positioned between the gripping section main body 84 and the proximal end cylinder section 66 is provided with an annular seal member 90 for preventing entry of foreign matter between the female screw section 78 and the male screw section 88.

The proximal end reduced diameter section 86 is provided at the proximal end section of the gripping section main body 84. The proximal end reduced diameter section 86 has an inner diameter that is less than the inner diameter of the proximal end section of the gripping section main body 84 and an outer diameter less than the outer diameter of the proximal end section of the gripping section main body 84. A cylindrical cover member 92 made of a resin material such as rubber is fitted to the proximal end reduced diameter section 86.

The attaching/detaching mechanism 52 is for attaching/detaching the catheter assembly 12 to/from the gripping member 50. The attaching/detaching mechanism 52 includes a holding member 94, a locking member 96, a biasing member 98, and a pressing member 100. Each of the holding member 94, the locking member 96, and the pressing member 100 can be made of the same material as the gripping member 50 described above.

The holding member 94 is for holding the locking member 96 at a predetermined position. The holding member 94 includes a cylindrical shaft section 102 inserted into the inner cylinder section 58 and a flange section 104 provided at the proximal end section of the shaft section 102. A recess 106 cut out in a direction orthogonal to the axial direction of the shaft section 102 is formed on a distal end side of an outer peripheral surface of the shaft section 102. The entire length of the shaft section 102 is longer than the entire length of the inner cylinder section 58.

The recess 106 is formed by a bottom surface 106a, a first side surface 106b connected to the distal end side with respect to the bottom surface 106a, and a second side surface 106c connected to the proximal end side with respect to the bottom surface 106a. The first side surface 106b is inclined in a distal end direction from the bottom surface 106a toward an outer peripheral surface of the shaft section 102. The second side surface 106c is inclined in the proximal end direction from the bottom surface 106a toward the outer peripheral surface of the shaft section 102. The flange section 104 protrudes radially outward from the proximal end section of the shaft section 102 and annularly extends in the circumferential direction.

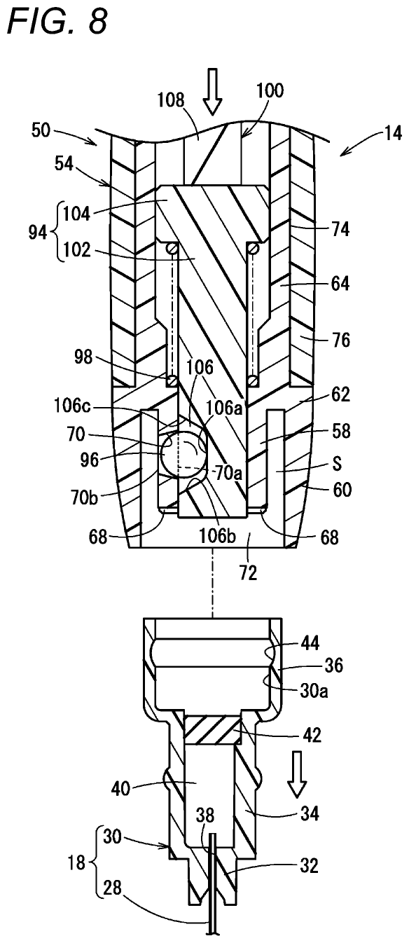

The locking member 96 is a ball (sphere) that is inserted into the hole 70 of the inner cylinder section 58 while being disposed in the recess 106 of the holding member 94. The locking member 96 protrudes radially outward (gap S between the inner cylinder section 58 and the outer cylinder section 60) from the outer opening 70b of the hole 70 while being in contact with the first side surface 106b forming the recess 106 (while being separated from the bottom surface 106a forming the recess 106). The locking member 96 does not protrude from the outer opening 70b of the recess 106 to the gap S while being in contact with the bottom surface 106a forming the recess 106 (state of FIG. 8).

The biasing member 98 biases the holding member 94 in the proximal end direction. The biasing member 98 is interposed between the connection section 62 and the flange section 104. As the biasing member 98, for example, a compression coil spring is used.

The pressing member 100 is disposed inside the gripping member 50 so as to be movable in the axial direction with respect to the gripping member 50. The pressing member 100 includes a rod section 108 abutting on the proximal end surface of the flange section 104, a stopper section 110 provided at the proximal end section of the rod section 108, and an operation section 112 extending in the proximal end direction from the stopper section 110.

The stopper section 110 protrudes radially outward from the proximal end section of the rod section 108 and extends annularly in the circumferential direction. The stopper section 110 is in contact with the proximal end reduced diameter section 86 to restrict the movement of the pressing member 100 in the proximal end direction with respect to the gripping member 50.

The operation section 112 protrudes in the proximal end direction with respect to the gripping member 50 via the inner hole of the proximal end reduced diameter section 86. In other words, the operation section 112 is positioned at the proximal end section (the second end section) of the gripping member 50.

In the catheter gripper 14, in the initial state, the holding member 94 is biased in the proximal end direction by the biasing member 98, and the stopper section 110 is in contact with the base proximal reduced diameter section 86. In this state, the distal end surface of the holding member 94 (shaft section 102) is positioned on the proximal end side with respect to the distal end of the inner cylinder section 58. In addition, the locking member 96 is in contact with a first side surface 106b and protrudes into the gap S through the hole 70 of the inner cylinder section 58.

Next, the use of the catheter system 10 configured in this manner will be described. A user prepares the catheter gripper 14, which is a reusable component, and the catheter assembly 12, which is a disposable component. Then, the user mounts the catheter assembly 12 on the catheter gripper 14.

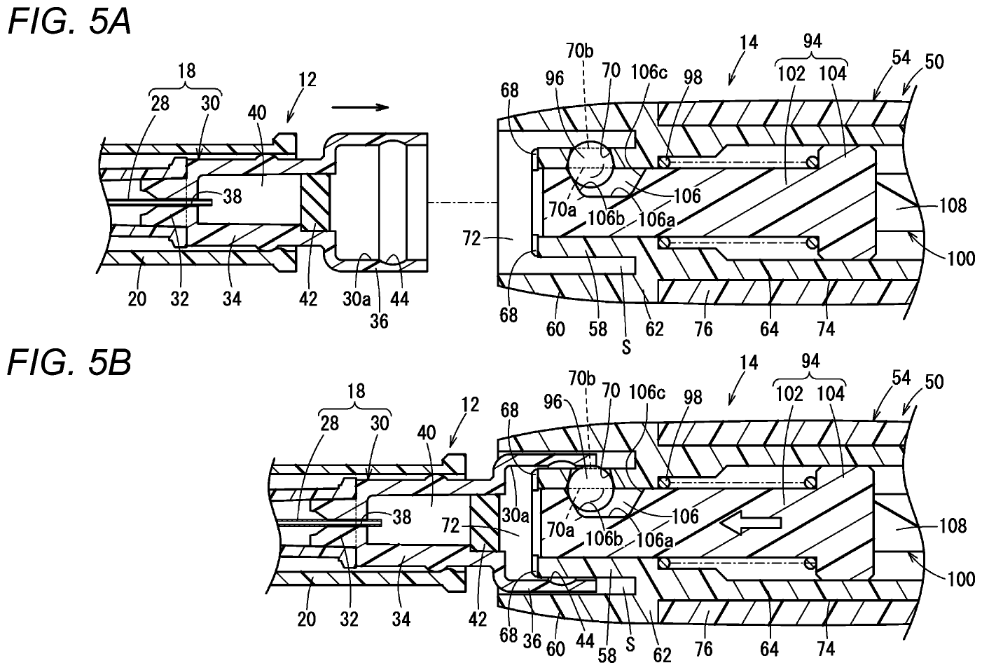

Specifically, as illustrated in FIG. 5A, the mounting section 36 of the catheter assembly 12 is pushed into the mounting hole 72 of the gripping member 50. In this way, the mounting section 36 is inserted into the gap S between the inner cylinder section 58 and the outer cylinder section 60. Then, when the mounting section 36 is in contact with the locking member 96, the locking member 96 is pressed against the bottom surface 106a forming the recess 106.

At this time, because the locking member 96 is in contact with the first side surface 106b, the holding member 94 is pushed in the distal end direction by the locking member 96. As a result, as illustrated in FIG. 5B, the holding member 94 moves in the distal end direction with respect to the gripping member 50 against a biasing force of the biasing member 98, and the locking member 96 is transferred to the bottom surface 106a side forming the recess 106.

When the mounting section 36 is further pushed into the proximal end direction with respect to the gripping member 50, the locking groove 44 of the mounting section 36 faces the hole 70 of the inner cylinder section 58. In this way, because the pressing against the locking member 96 by the mounting section 36 is released, the holding member 94 is pushed into the proximal end direction by the biasing force of the biasing member 98.

As a result, as illustrated in FIG. 6, the locking member 96 is pushed into the proximal end direction by the first side surface 106b forming the recess 106, protrudes from the hole 70 of the inner cylinder section 58 to the gap S, and is pressed against the wall surface forming the locking groove 44 of the mounting section 36. As a result, the movement of the mounting section 36 in the axial direction with respect to the catheter gripper 14 is prevented. That is, the mounting of the catheter assembly 12 on the catheter gripper 14 is completed.

Subsequently, the user removes the protector 20, and punctures the blood vessel with the inner needle 28 and the catheter shaft 22 while gripping the gripping member 50. In this way, blood in a blood vessel flows into the chamber 40 of the needle hub 30 via the inner cavity 28a of the inner needle 28. At this time, the air in the chamber 40 is pushed out from the chamber 40 to the proximal end side via the filter member 42 by blood. The air pushed out to the proximal end side of the filter member 42 is discharged to the outside of the outer cylinder section 60 via the air vent flow path 73 (air discharge groove 68, first gap S1, second gap S2, third gap S3). Therefore, blood smoothly flows into the chamber 40.

The user can recognize that the distal end opening of the inner needle 28 is reliably positioned in the blood vessel by visually recognizing that the blood has flowed into the chamber 40 (flashback) via the needle hub 30.

Thereafter, the user retracts the catheter gripper 14 in a state in which the catheter shaft 22 is indwelled in the blood vessel to remove the needle member 18 with respect to the catheter assembly 12. In this state, only the needle member 18 is mounted on the catheter gripper 14.

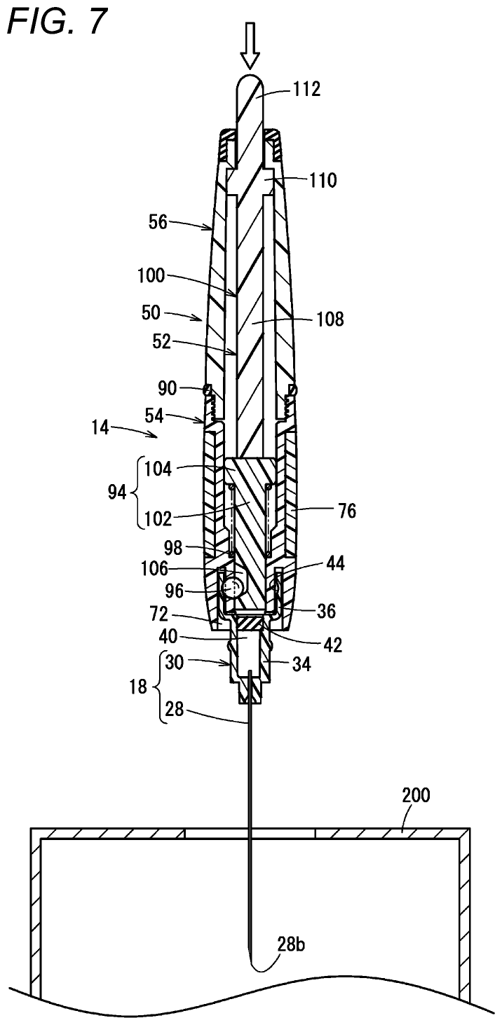

Subsequently, as illustrated in FIG. 7, the user directs the needle tip 28b downward (in the direction of gravity) and presses the operation section 112 in the distal end direction while being placed in a disposal box 200. In this way, as illustrated in FIG. 8, the biasing member 98 (compression coil spring) is compressed, and the holding member 94 moves in the distal end direction with respect to the gripping member 50. When the holding member 94 moves in the distal end direction, because the locking member 96 is retracted so as to be in contact with the bottom surface 106a forming the recess 106, the pressing force of the locking member 96 against the wall surface forming the locking groove 44 of the mounting section 36 is released.

Then, because the distal end surface of the holding member 94 protrudes more toward the distal end side than the inner cylinder section 58 and presses the proximal end section of the needle hub body 34 in the distal end direction, the needle member 18 is pushed out in the distal end direction. Therefore, the user can safely remove the needle member 18 from the catheter gripper 14 without being in contact with the needle member 18 and discard the needle member in the disposal box 200.

In this case, the present embodiment has the following effects.

The catheter system 10 includes the catheter assembly 12 and the catheter gripper 14 attachable to and detachable from the catheter assembly 12. The catheter gripper 14 includes the gripping member 50 extending in one direction and the attaching/detaching mechanism 52 for attaching/detaching the proximal end section of the needle hub 30 to/from a first end section (distal end section) of the gripping member 50.

According to such a configuration, because the catheter assembly 12 is attachable to and detachable from the catheter gripper 14, the catheter assembly 12 can be configured as a disposable product and the catheter gripper 14 can be configured as a reusable product. As a result, it is not necessary to provide the gripping portion in the catheter assembly 12 to be discarded after one use and, thus, the entire length L1 of the catheter assembly 12 can be made relatively short. Therefore, the waste (catheter assembly 12) can be reduced in size. In addition, because the number of catheter grippers 14, which are reusable products, is usually less than the number of catheter assemblies 12, which are disposable products, the storage space can be reduced.

The catheter gripper 14 includes an operation section 112 for performing an operation of removing the needle hub 30 from the gripping member 50.

According to such a configuration, the user can remove the catheter assembly 12 from the catheter gripper without being in direct contact with the catheter assembly 12 by operating the operation section 112.

The operation section 112 is positioned at the second end (proximal end) of the gripping member 50.

According to such a configuration, it is possible to further suppress the user from being in contact with the catheter assembly 12 when operating the operation section 112.

The first end section (distal end section) of the gripping member 50 is provided with the mounting hole 72, and the attaching/detaching mechanism 52 is formed so that the needle hub 30 is mounted on the gripping member 50 by pushing the proximal end section (mounting section 36) of the needle hub 30 into the mounting hole 72.

According to such a configuration, the needle hub 30 can be easily mounted on the gripping member 50.

The attaching/detaching mechanism 52 includes the pressing member 100 for pressing the proximal end section of the needle hub 30 mounted on the gripping member 50 in the distal end direction of the needle hub 30, and the needle hub 30 is removed from the gripping member 50 by being pressed in the distal end direction of the needle hub 30 by the pressing member 100.

According to such a configuration, the needle hub 30 can be removed from the gripping member 50 by a simple operation of pressing the pressing member 100.

The gripping member 50 is provided with the air vent flow path 73 for discharging air, which is guided from the inner cavity 30a of the needle hub 30 to the gripping member 50 side, to the outside in a state in which the proximal end section of the needle hub 30 is mounted on the gripping member 50.

According to such a configuration, because the air in the inner cavity 30a of the needle hub 30 can be discharged to the outside via the air vent flow path 73, it is possible to efficiently introduce a biological fluid (blood) into the inner cavity 30a of the needle hub 30.

The inner cavity 30a of the needle hub 30 is provided with a filter member 42 that allows the air to flow but blocks the blood from flowing.

According to such a configuration, it is possible to prevent the blood introduced into the inner cavity 30a of the needle hub 30 from leaking to the outside by the filter member 42.

The needle hub 30 is provided with the chamber 40 into which the blood guided from the inner needle 28 flows, and at least a part of the needle hub 30 has transparency so that the blood flowing into the chamber 40 can be visually recognized from the outside of the needle hub 30.

According to such a configuration, it is possible to easily recognize that the distal end opening of the inner needle 28 is positioned in the blood vessel by visually recognizing the blood flowing into the chamber 40 of the needle hub 30.

The ratio of the entire length L3 of the needle hub 30 to the entire length L2 of the catheter hub 24 is 150% or less.

According to such a configuration, the entire length of the catheter assembly 12 can be made relatively short.

The mounting section 36 is provided with the locking groove 44 in which the locking member 96 of the catheter gripper 14 can be engaged for locking.

According to such a configuration, the catheter assembly 12 can be mounted on the catheter gripper 14 with a simple configuration.

The mounting section 36 is formed in an annular shape, and the locking groove 44 is provided on the inner peripheral surface of the mounting section 36.

According to such a configuration, because the locking member 96 of the catheter gripper 14 can be disposed inside the mounting section 36, the configuration of the catheter gripper 14 can be simplified.

The needle hub 30 has the annular needle hub body 34 adjacent to the distal end side of the mounting section 36, and the inner diameter of the needle hub body 34 is less than that of the mounting section 36.

According to such a configuration, the needle hub 30 can be easily removed from the catheter gripper 14 by pressing the attaching/detaching mechanism 52 of the catheter gripper 14 against the proximal end surface of the needle hub body 34.

The present invention is not limited to the above-described embodiments, and various modifications can be made without departing from the gist of the present invention.

In the catheter gripper 14, the position where the operation section 112 is provided for the gripping member 50 can be appropriately changed. That is, the operation section 112 may be positioned at the intermediate section or the distal end section (first end section) of the gripping member 50.

The above embodiments are summarized as follows.

According to one embodiment, a catheter system 10 includes the catheter assembly 12 that includes the inner needle 28 having the needle tip 28b at the distal end thereof, the hollow needle hub 30 fixed to the proximal end section of the inner needle, the catheter shaft 22 into which the inner needle is inserted, and the catheter hub 24 having the inner needle inserted thereinto and fixed to the proximal end section of the catheter shaft, and the catheter gripper 14 that is attachable to and detachable from the catheter assembly. The catheter gripper includes the gripping member 50 that extends in one direction, and the attaching/detaching mechanism 52 that attaches/detaches the proximal end section of the needle hub to/from a first end section of the gripping member.

In the above-described catheter system, the attaching/detaching mechanism may include the operation section 112 for performing an operation of removing the needle hub from the gripping member.

In the above-described catheter system, the operation section may be positioned at the second end section of the gripping member.

In the above-described catheter system, the first end section of the gripping member may be provided with the mounting hole 72, and the attaching/detaching mechanism may be formed so that the needle hub is mounted on the gripping member by pushing the proximal end section of the needle hub into the mounting hole.

In the above-described catheter system, the attaching/detaching mechanism may include the pressing member 100 for pressing the proximal end section of the needle hub mounted on the gripping member in the distal end direction of the needle hub, and the needle hub may be removed from the gripping member by being pressed in the distal end direction of the needle hub by the pressing member.

In the above-described catheter system, the gripping member may be provided with the air vent flow path 73 for discharging the air, which is guided from the inner cavity 30a of the needle hub to the gripping member side, to the outside in the state in which the proximal end section of the needle hub is mounted on the gripping member.

In the above-described catheter system, the inner cavity of the needle hub may be provided with the filter member 42 that allows the air to flow but blocks the blood from flowing.

In the above-described catheter system, the needle hub may be provided with the chamber 40 into which the blood guided from the inner needle flows, and at least a part of the needle hub may have transparency so that the blood flowing into the chamber can be visually recognized from the outside of the needle hub.

According to another embodiment, a catheter assembly includes the inner needle that has the needle tip at the distal end thereof, the hollow needle hub that is fixed to the proximal end section of the inner needle, the catheter shaft into which the inner needle is inserted, and the catheter hub that has the inner needle inserted thereinto and is fixed to the proximal end section of the catheter shaft. The proximal end section of the needle hub is provided with the mounting section 36 for mounting on the catheter gripper.

In the above-described catheter assembly, the ratio of the entire length L3 of the needle hub to the entire length L2 of the catheter hub may be 150% or less.

In the above-described catheter assembly, the mounting section may be provided with the locking groove 44 in which a locking member 96 of the catheter gripper can be engaged for locking.

In the above-described catheter assembly, the mounting section may be formed in an annular shape, and the locking groove may be provided on the inner peripheral surface of the mounting section.

In the above-described catheter assembly, the needle hub may have the annular needle hub body 34 adjacent to the distal end side of the mounting section, and the inner diameter of the needle hub body may be less than that of the mounting section.

According to another embodiment, a catheter gripper is attachable to and detachable from the catheter assembly that includes the inner needle that has the needle tip at a distal end thereof, the hollow needle hub that is fixed to the proximal end section of the inner needle, the catheter shaft into which the inner needle is inserted, and the catheter hub that has the inner needle inserted thereinto and is fixed to the proximal end section of the catheter shaft. The catheter gripper includes the gripping member that extends in one direction, and the attaching/detaching mechanism that attaches/detaches the proximal end section of the needle hub to/from a first end section of the gripping member.

What is claimed is:

1. A catheter system comprising:
a catheter assembly that comprises:
an inner needle having a needle tip at a distal end thereof,
a hollow needle hub fixed to a proximal end section of the inner needle,
a catheter shaft into which the inner needle is inserted, and
a catheter hub having the inner needle inserted thereinto, wherein the catheter hub is fixed to a proximal end section of the catheter shaft; and
a catheter gripper that is attachable to and detachable from the catheter assembly, wherein the catheter gripper comprises:
a gripping member, and
an attaching/detaching mechanism configured to attach and detach a proximal end section of the needle hub to and from a first end section of the gripping member.

2. The catheter system according to claim 1, wherein:
the attaching/detaching mechanism comprises an operation section for performing an operation of removing the needle hub from the gripping member.

3. The catheter system according to claim 2, wherein:
the operation section is positioned at an second end section of the gripping member.

4. The catheter system according to claim 1, wherein:
the first end section of the gripping member comprises a mounting hole, and
the needle hub is mountable on the gripping member by a proximal end section of the needle hub being pressed into the mounting hole.

5. The catheter system according to claim 1, wherein:
the attaching/detaching mechanism comprises a pressing member for pressing a proximal end section of the needle hub mounted on the gripping member in a distal end direction of the needle hub, and
the needle hub is removable from the gripping member by being pressed in the distal end direction of the needle hub by the pressing member.

6. The catheter system according to claim 1, wherein:
the gripping member comprises an air vent flow path for discharging air, the air vent flow path being configured such that, in a state in which the proximal end section of the needle hub is mounted on the gripping member, the air vent flow path guides the air from an inner cavity of the needle hub to a gripping member side, to an outside.

7. The catheter system according to claim 6, wherein:
the inner cavity of the needle hub comprises a filter member that allows the air to flow but blocks blood from flowing.

8. The catheter system according to claim 1, wherein:
the needle hub comprises a chamber into which blood guided from the inner needle flows, and
at least a part of the needle hub has transparency so that the blood flowing into the chamber is visually recognizable from an outside of the needle hub.

9. A catheter assembly comprising:
an inner needle that has a needle tip at a distal end thereof;
a hollow needle hub that is fixed to a proximal end section of the inner needle;
a catheter shaft into which the inner needle is inserted; and
a catheter hub that has the inner needle inserted thereinto and is fixed to a proximal end section of the catheter shaft,
wherein a proximal end section of the needle hub comprises a mounting section for mounting the needle hub on a catheter gripper.

10. The catheter assembly according to claim 9, wherein:
a ratio of an entire length of the needle hub to an entire length of the catheter hub is 150% or less.

11. The catheter assembly according to claim 9, wherein:
the mounting section comprises a locking groove in which a locking member of the catheter gripper is engageable for locking.

12. The catheter assembly according to claim 11, wherein:
the mounting section is formed in an annular shape, and
the locking groove is located on an inner peripheral surface of the mounting section.

13. The catheter assembly according to claim 9, wherein:
the needle hub comprises an annular needle hub body adjacent to a distal end side of the mounting section, and
an inner diameter of the needle hub body is less than an inner diameter of the mounting section.

14. A catheter gripper attachable to and detachable from a catheter assembly that includes an inner needle that has a needle tip at a distal end thereof, a hollow needle hub that is fixed to a proximal end section of the inner needle, a catheter shaft into which the inner needle is inserted, and a catheter hub that has the inner needle inserted thereinto, wherein the catheter hub is fixed to a proximal end section of the catheter shaft, the catheter gripper comprising:
a gripping member; and
an attaching/detaching mechanism configured to attach and detach a proximal end section of the needle hub to and from a first end section of the gripping member.
